(19) United States
(12) Patent Application Publication
(10) Pub. No.: US 2004/0023389 A1
(43) Pub. Date: Feb. 5, 2004

(54) Adenoviral vectors having nucleic acids encoding immunomodulatory molecules

(76) Inventors: Abraham Scaria, Framingham, MA (US); Samuel Wadsworth, Shrewsbury, MA (US)

Correspondence Address: GENZYME CORPORATION, LEGAL DEPARTMENT, 15 PLEASANT ST CONNECTOR, FRAMINGHAM, MA, 01701-9322, US

(21) Appl. No.: 10/292,893

(22) Filed: Nov. 12, 2002

Related U.S. Application Data
(63) Continuation of application No. 09/552,511, filed on Apr. 19, 2000, now abandoned.
Application No. 09/552,511 is a continuation of application No. 09/496,248, filed on Feb. 2, 2000, now abandoned.
(60) Provisional application No. 60/130,415, filed on Apr. 21, 1999.

(51) Int. Cl. A61K 48/00; C12N 7/00; C12N 15/861

(52) U.S. Cl. 435/456; 435/235.1; 435/320.1; 424/93.2

(57) ABSTRACT

The invention relates to recombinant adenoviral vectors for use in delivering a nucleic acid(s) encoding an immunomodulatory molecule(s) to the cells of an individual that allows the vector to reduce or evade the host immune response from the cells of said individual. These vectors could be used to induce tolerance to an adenovirus antigen or transgenic products by transduction of antigen-presenting cells of an individual and/or increase the half-life of antigen-presenting cells in order to enhance immune response against tumor antigens.

The invention further relates to recombinant adenoviral vectors for use in delivering desired therapeutic transgenes to cells in patients, said vectors containing at least one nucleic acid encoding an immunomodulatory molecule that allow the vectors containing said nucleic acid(s) to reduce or evade the host antiviral immune response to the adenovirus and one or more transgenes. These vectors are capable of increased persistence in the individual to whom they are administered, thereby facilitating longer term administration of transgenes and reduced immunologic response upon administration. The invention also relates to methods for the use of such vectors in delivering transgenes to patients for therapeutic uses.

### INTRODUCTION

The invention relates to recombinant adenoviral vectors that can be used to deliver a nucleic acid encoding an immunomodulatory molecule(s) to the cells of an individual. The nucleic acid encoding the immunomodulatory molecule allows the vector to reduce or evade an immune response to the vector or the cells harboring the vector. The vectors may be used to induce tolerance to an adenovirus antigen and/or biologically active transgene product in antigen-presenting cells of an individual to whom they are administered, increase the half-life of cells in the body that have taken up the vector and expressed the antigen and/or transgene product, e.g. antigen-presenting cells.

The invention further relates to recombinant adenoviral vectors that can be used to deliver a desired transgene to cells of an individual, said vectors containing at least one nucleic acid encoding an immunomodulatory molecule that allow such vectors to reduce or evade the host antiviral immune response to the adenovirus and/or the transgene. These vectors can provide increased persistence in the individual to whom they are administered, thereby reducing the need for multiple readministration, as well as reduced immunologic response upon administration or readministration. The invention also relates to methods for the use of such vectors in delivering genes to cells of an individual for expression therein.

### BACKGROUND OF THE INVENTION

The ability to deliver a transgene to a target cell or tissue and have it expressed therein to produce a desired phenotypic effect depends on the development of gene transfer vehicles that can safely and efficiently deliver an exogenous nucleic acid (transgene) to the recipient cell. To this end, most efforts have focused on the use of virus-derived vectors in order to exploit the natural ability of a virus to deliver its genetic content to a target cell.

Early strategies have focused on retroviral vectors which have been the vectors of choice to deliver therapeutic transgenes for gene therapy because of their ability to integrate into the cellular genome. However, the disadvantages of retroviral vectors have become evident, including their tropism for dividing cells only, the possibility of insertional mutagenesis in the host genome upon integration of the vector nucleic acid into the host cell genome, decreased expression of the transgene over time, rapid inactivation of retroviruses by the serum complement system, and the possibility of generating replication-competent retroviruses (Jolly, D., Cancer Gene Therapy 1:51-64, 1994; Hodgson, C. P., Bio/Technology 13:222-225, 1995).

Adenovirus, a nuclear double-stranded DNA virus with a genome of about 36 kb, has been well-characterized through studies in classical genetics and molecular biology (Horwitz, M. S., “Adenoviridae and Their Replication,” in Virology, 2nd edition, Fields et al., eds., Raven Press, New York, 1990). The adenoviral genome is classified into early (known as E1-E4) and late (known as L1-L5) transcriptional units, referring to the generation of two temporal classes of viral proteins. The demarcation between these events is viral DNA replication.

The cloning capacity of an adenovirus vector is proportional to the size of the adenovirus genome present in the vector. For example, a cloning capacity of about 8 kb can be created from the deletion of certain regions of the virus genome dispensable for virus growth, e.g., E3, and the deletion of a genomic region such as El whose function may be restored in trans from 293 cells (Graham, F. L., J. Gen. Virol. 36:59-72, 1977) or A549 cells (Imler et al., Gene Therapy 3:75-84, 1996). Such E1-deleted vectors are rendered replication-defective. The upper limit of vector DNA capacity for optimal carrying capacity is about 105%-108% of the length of the wild-type genome. Further adenovirus genomic modifications are possible in vector design using cell lines which supply other viral gene products in trans, e.g., complementation of E2a (Zhou et al., J. Virol. 70:7030-7038, 1996), complementation of E4 (Krougliak et al., Hum. Gene Ther. 6:1575-1586, 1995; Wang et al., Gene Ther. 2:775-783, 1995), or complementation of protein IX (Caravokyri et al., J. Virol. 69:6627-6633, 1995; Krougliak et al., Hum. Gene Ther. 6:1575-1586, 1995).

Adenovirus-derived vectors have several advantages, including tropism for both dividing and non-dividing cells, lessened pathogenic potential, ability to replicate to high titer for preparation of vector stocks, and the potential to carry large DNA inserts (Berkner, K. L., Curr. Top. Micro. Immunol. 158:39-66, 1992; Jolly, D., Cancer Gene Therapy 1:51-64, 1994). The cloning capacity of an adenoviral vector is a factor of the deletion of certain regions of the virus genome dispensable for virus growth (e.g., E3) or deletions of regions whose function is restored in trans from a packaging cell line (e.g., E1, with complementation of E1 functions by 293 cells (Graham, F. L., J. Gen. Virol. 36:59-72, 1977). The upper limit for optimal packaging may be extended to about 105%-108% of wild-type adenoviral genome length for increased carrying capacity in the vector.

Genes that have been expressed to date by adenoviral vectors include p53 (Wills et al., Human Gene Therapy 5:1079-188, 1994); dystrophin (Vincent et al., Nature Genetics 5:130-134, 1993); erythropoietin (Descamps et al., Human Gene Therapy 5:979-985, 1994); ornithine transcarbamylase (Stratford-Perricaudet et al., Human Gene Therapy 1:241-256, 1990; We et al., J. Biol. Chem. 271:3639-3646, 1996); adenosine deaminase (Mitani et al., Human Gene Therapy 5:941-948, 1994); interleukin-2 (Haddada et al., Human Gene Therapy 4:703-711, 1993); al-antitrypsin (Jaffe et al., Nature Genetics 1:372-378, 1992); thrombopoietin (Ohwada et al., Blood 88:778-784, 1996); cytosine deaminase (Ohwada et al., Hum. Gene Ther. 7:1567-1576, 1996); human alpha-galactosidase A (PCT No. PCT/US98/22886), human beta-galactosidase (Arthur et al., Cancer Gene Therapy 4:17-25, 1997); interleukin-7 (Arthur et al, Cancer Gene Therapy 4:17-25, 1997); a Herpes Simplex Virus thyrnidine kinase gene (U.S. Pat. No. 5,763,415) and cystic fibrosis transmembrane conductance regulator (CFTR) (U.S. Pat. Nos. 5,670,488 and 5,882,877; Zabner et al., J. Clin. Invest. 97:1504-1511, 1996).

The tropism of adenoviruses for cells of the respiratory tract has particular relevance to the use of adenoviral vectors in therapy for cystic fibrosis (CF), which is the most common autosomal recessive disease in Caucasians. Individuals with CF sustain pulmonary dysfunction resulting from mutations in the transmembrane conductance regulator (CFTR) gene that disturb the proper functioning of the cAMP-regulated Cl⁻ channel in airway epithelia (Zabner, J. et al., Nature Genetics 6:75-83, 1994). Adenoviral vectors which carry the CFTR gene have been developed (Rich, D. et al., Human Gene Therapy 4:461-476, 1993) and studies have shown that these vectors can deliver CFTR to the airway epithelia of CF patients (Zabner, J. et al., Cell 75:207-216, 1993; Zabner, J. et al., J. Clin. Invest. 97:1504-1511, 1996), the airway epithelia of cotton rats and primates (Zabner, J. et al., Nature Genetics 6:75-83, 1994), and the respiratory epithelium of CF patients (Crystal, R. G. et al., Nature Genetics 8:42-51, 1994). Recent studies have shown that administering an adenoviral vector containing a DNA encoding CFTR to airway epithelial cells of CF patients can restore a functioning chloride ion channel in the treated epithelial cells (Zabner et al., J. Clin. Invest. 97:1504-1511, 1996).

The preclinical studies and clinical trials so far conducted using adenoviral vectors for gene therapy, however, have suggested that administration of these vectors is associated with an antiviral host immune response that may limit positive results. The immune system functions to protect the body against infection by detecting the presence of macromolecules that are “foreign”. These foreign molecules can be viral or other parasite proteins; these proteins can be present in the circulatory system of the body or present only within certain cells in the body. This form of immunity is based on the ability of the body to recognize foreign agents and to respond with the production of antibodies, so-called humoral immunity, or lymphocytes with killing activity, so-called cellular immunity. The effectors of humoral and cellular immunity have a high degree of specificity and immunological memory. In addition, more general defense mechanisms that do not depend on previous exposure of the body to the foreign agent also exist; this is referred to as innate immunity. The ability of macrophages to engulf particles, cytokine responses as part of the inflammatory response and the recognition of bacterial DNA methylation patterns are all examples of innate immunity.

Viruses are examples of infectious agents to which the body responds with both innate and specific immunity. Viral structural proteins can stimulate antibody responses which can neutralize virus infectivity, and viral proteins synthesized within the cell can stimulate a cellular immune response which can target virus-infected cells for destruction. Moreover, viruses can provoke a non-specific inflammatory response as well. Vectors based on viruses can, in theory, be affected by specific and innate immune responses, depending on whether viral genes are retained within the vector and whether the viral genes are expressed at levels sufficient to provoke an immune response. The level at which viral genes are expressed is potentially very significant, since inflammatory and immune responses are proportional to dose. A low dose of antigen can escape detection by the body while a large dose is far more likely to be detected and to provoke a response. Some viral-based vectors are believed to present less of an immunological problem. AAV and retroviral vectors, for example, in most instances have had all viral genes deleted, thus eliminating the possibility that a cellular immune response will be generated against viral gene products. These vectors can, nonetheless, stimulate antibody responses to the virion coat proteins if sufficient amounts of vector are introduced into the body.

Conventional E1-deleted adenovirus vectors usually retain numerous viral genes, since vigorous expression of the remaining genes is dependent on the activity of the deleted E1 gene products (provided in trans by producer cells). Furthermore, since adenovirus does not integrate into the cell genome, host immune responses that destroy virions or virus-infected cells have the potential to limit long-term delivery and expression of a transgene in a target cell using a vector that elicits the production of immunogenic molecules (especially proteins) that can trigger a pathological inflammatory response to the vector and/or the transgene and/or the target cell which may destroy or adversely affect the adenovirus-infected cells (Yang et al., Nature Genetics 7:362-369, 1994; Yang et al., Proc. Natl. Acad. Sci. 91:4407-4411, 1994; Zsengeller et al., Hum. Gene Ther. 6:457-467, 1995; Worgall et al., Hum. Gene Ther. 8:37-44, 1997; Kaplan et al., Hum. Gene Ther. 8:45-56, 1997). This adverse immunologic response poses a serious obstacle for high dose administration of an adenoviral vector or for its repeated or long-term administration for transgene expression (Crystal, R., Science 270:404-410, 1995).

Immunogenic reactions by a host to adenovirus infection include, inter alia, the generation of cytotoxic T-lymphocytes (CTL) which lyse infected cells displaying a viral antigen, cytolysis of virus-infected cells by tumor necrosis factor (TNF), synthesis of interferons, induction of apoptosis, and other immunologic mechanisms (Smith, G. L., Trends Microbiol. 2:81-88, 1994).

In addition, it is essential to consider the effect of the transgene's product on the body's response to vector administration when the transgene product is mutated or absent in the target cells. An immune response to a transgene product can occur regardless of the vector class, whether it is viral or nonviral. For example, individuals with blood clotting disorders such as hemophilia have mutations in their factor VIII or factor IX genes. Some patients make defective protein or small amounts of active protein, in which case, their immune systems “see” the protein (Factor VIII or IX) as a self protein. Other individuals have completely non-functional genes for clotting factors so they do not normally produce the relevant protein. As a result, their immune system has never “seen” the protein. However, if a transgene encoding factor VIII or factor IX is supplied by a vector to the cells of an individual that expresses some clotting factor, then there is a low probability that patient will respond immunologically to the protein encoded by the vector. However, if a transgene encoding factor VIII or factor IX is supplied by a vector to an individual that expresses no clotting factor, then there is a high probability that this individual will respond immunologically to the protein encoded by the vector. This immune response can be at the humoral and/or cellular level; that is antibodies can arise which can clear the protein from the blood and/or cytotoxic lymphocytes can arise which can destroy the cells containing the vector and expressing the therapeutic protein.

In order to circumvent the host immune response which limits the persistence of transgene expression, various strategies have been employed, generally involving either the modulation of the immune response itself or the engineering of a vector that decreases the immune response. The administration of immunosuppressive agents, together with vector administration, has been shown to prolong transgene persistence (Fang et al., Hum. Gene Ther. 6:1039-1044, 1995; Kay et al., Nature Genetics 11:191-197, 1995; Zsellenger et al., Hum. Gene Ther. 6:457-467, 1995; Scaria et al., Gene Therapy 4:611-617, 1997; WO98/08541).

Modifications to genomic adenoviral sequences contained in the recombinant vector have been attempted in order to decrease the host immune response (Yang et al., Nature Genetics 7:362-369, 1994; Lieber et al., J. Virol. 70:8944-8960, 1996; Gorziglia et al., J. Virol. 70:4173-4178; Kochanek et al., Proc. Natl. Acad. Sci. USA 93:5731-5736, 1996; Fisher et al., Virology 217:11-22, 1996). The adenovirus E3 gp19K protein can complex with MHC Class I antigens and retain them in the endoplasmic reticulum, which prevents cell surface presentation and killing of infected cells by cytotoxic T-lymphocytes (CTLs) (Wold et al., Trends Microbiol. 2:437-443, 1994), suggesting that its presence in a recombinant adenoviral vector may be beneficial. The lack of persistence in the expression of adenoviral vector-delivered transgenes may also be due to limitations imposed by the choice of promoter or transgene contained in the transcription unit (Guo et al., Gene Therapy 3:802-801, 1996; Tripathy et al., Nature Med. 2:545-550, 1996). Further optimization of minimal adenoviral vectors for persistent transgene expression in target cells and tissues also involves the design of expression control elements, such as promoters, which confer persistent expression to an operably linked transgene. Promoter elements, which function independently of particular viral genes to confer persistent expression of a transgene, allow the use of vectors containing reduced viral genomes (see, e.g., U.S. Pat. No. 5,882,877).

Many viruses have evolved mechanisms to counter an antiviral response of the host, such as the ability to express viral genes encoding immunomodulatory proteins. These proteins are a heterogenous group, characterized by their ability to interfere with an antiviral host immune response through a variety of mechanisms. For example, viral proteins of poxviruses and herpesvirus are able to block complement activation. Adenoviruses, poxviruses, herpesvirus and HIV-1 each encode proteins which enable the virus to resist interferon-mediated inhibition of viral replication (Smith, G. L., Trends Microbiol. 2:81-88, 1994). The B15R gene of vaccinia virus encodes a protein capable of binding interleukin-1, thereby reducing its antiviral effect (Spriggs et al., Cell 71:145-152, 1992; Alcami et al., Cell 71:153-167, 1992).

The crmA gene of cowpox virus encodes a serpin, which is a specific inhibitor of the interleukin-1β converting enzyme (Ray et al., Cell 69:597-604, 1992) and which inhibits apoptosis of virus-infected cells (Tewari et al., J. Biol. Chem. 270:3255-3260, 1995). The BCRF1 gene of Epstein-Barr virus encodes a protein that is homologous to interleukin-10, which inhibits the synthesis of interferon-γ (Moore et al., Science 248: 1230-1234, 1990; Hsu et al., Science 250:830-832, 1990). The ICP47 gene of herpes simplex virus encodes a protein which blocks presentation of viral peptides to MHC class I-restricted cells (Hill et al., Nature 375:411-415, 1995). Cytomegalovirus (CMV) contains the US11, gene that encodes an endoplasmic reticulum (ER) transmembrane protein which transports newly synthesized MHC class I products from the ER to the cytosol for proteolytic degradation (Wiertz et al., Cell 84:769-779, 1996).

Adenoviral vectors have been designed which specifically retain the viral E3 region in order to exploit the potential of the genes encoded by this region, such as gp19K, to reduce host immune response to adenovirus-infected cells (Rich et al., Human Gene Therapy 4:461-476, 1993). There have also been suggestions to incorporate non-adenoviral genes into adenoviral vectors that code for proteins that suppress the presentation of Class I antigens in order to minimize the immune response to adenoviral antigens.

The p35 gene of baculovirus encodes a protein which blocks apoptosis of baculovirus-infected cells (Clem et al., Science 254:1388-1389, 1991; Hershberger et al., J. Virol. 68:3467-3477, 1994; Bump et al., Science 269:1885-1888, 1995; Xue et al., Nature 377: 248-251, 1995).

Adenovirus encodes the E1B 19K and the E3 14.7K, 14.5K, and 10.4K proteins which are able to protect infected cells from TNF-induced cytolysis. As discussed above, the adenovirus E3 gp19K protein can complex with MHC Class I antigens and retain them in the endoplasmic reticulum, which prevents cell surface presentation and killing of infected cells by cytotoxic T-lymphocytes (CTLs) (Wold et al., Trends Microbiol. 2:437-443, 1994).

Immunomodulatory proteins are also produced by mammalian cells. For example, cytotoxic lymphocyte cytoplasmic serpin proteinase inhibitor 9 (P-I-9) is a protein that is expressed at high levels in the cytoplasm of cytotoxic lymphocytes. P-I-9 is a proteinase inhibitor that efficiently inhibits killing induced by granzyme B in vitro (Bird et al., Mol. Cell. Biol. 18:6387-6398, 1998 and Sun et al., J. Biol. Chem. 271:27802-27809, 1996). It has been hypothesized that P-I-9 protects the cytotoxic lymphocytes against premature death triggered by misdirected or miscompartmentalized granzyme B (Bird et al., Mol. Cell. Biol. 18:6387-6398, 1998).

The mammalian fas/CD95 ligand (fasL) cell surface protein induces apoptosis of T cells responding to foreign antigens in transplantation, so that donor tissue expressing fasL is protected against rejection by the host immune response, even in mismatched grafts (Griffith et al., Science 270:1189-1192, 1995; Bellgrau et al., Nature 377:630-632, 1995).

One strategy used to overcome the immune response to adenoviral vector proteins as well as transgene products is the induction of peripheral T-cell tolerance to both the adenoviral vector and the transgene products which, in many cases, may be a neoantigen in the patient. It is known that activation-induced cell death in T cells, in which apoptosis of the T cells is mediated by upregulation of fas and fas ligand is responsible for down regulation of the T-cell response and T cell homeostasis (Watanabe-Fukunaga et al., Nature:314-317, 1992; Zhou et al., J. Exp. Med. 176:1063-1072, 1992; Nagata, Adv. Immunol. 57:129-144, 1994, and Dhein et al., Nature 373:438-441, 1995). Recently, it has been shown that adenovirus mediated fasL expression in balloon-injured rat carotid artery, even in animals pre-immunized with adenovirus vector, resulted in effective inhibition of neointima formation as well as protection of the Ad/fasL infected cells from immune destruction (Sata et al., Proc. Nati. Acad. USA 95:1213-1217, 1998).

One mechanism of tolerance is the use of the natural occurrence of immune privileged sites. Clonal deletion of antigen-specific T cells, mediated by apoptosis of T cells via the fas-fasL pathway has been shown to be an important mechanism in creating immune-privileged sites, as well as in the maintenance of peripheral tolerance, and in the prevention of graft rejection (Nagata, Adv. Immunol. 57:129-144, 1994; Dhein et al., Nature 373:438-441, 1995; Bellgrau et al., Nature 377:630-632, 1995; Griffith et al, Science 270:1189-1192, 1995 and Lau et al, Science 273:109-112, 1996). Insertion of adenovirus at these sites results in tolerance to adenovirus and its transgene product. The injection of E1 deleted adenovirus injected into the subretinal space resulted in minimal cellular and humoral immune response (Bennet et al., Hum. Gene Therap. 8:1625-1634, 1996).

Recently, it has been disclosed that pretreatment of mice with an antigen-presenting line expressing Ad/fasL can induce Ad-specific T cell tolerance (WO98/52615, WO98/51340 and Zhang et al., Nature BioTechnology 16:1045-1049). Upon subsequent intravenous administration of an Ad/lacZ vector, persistent expression of the lacZ gene was achieved in the liver for 50 days. However, there are two major drawbacks to this procedure. The first is that the method used for expression of the fasL gene in the antigen-presenting cell line involves coinfection with two different adenoviral vectors, AdLoxpfasL (Zhang et al., J. Virol. 72:2483-2490, 1998) and AxCANCre (Kanegae et al, Nucl. Acids Res. 23:3816-3821, 1995). In this method, AxCANCre expresses the Cre recombinase and is required to turn on expression of the fasL gene from the AdLoxpfasL vector. Thus, an extra component besides the vector which contains the fas gene is required for its effective delivery. Ad vectors constitutively expressing fasL cannot be grown to high titers, because fasL induces apoptosis of the 293 cells used to propagate the Ad vectors (Larregina et al., Gene Ther. 5:563-568, 1998). Secondly, the antigen-presenting cell line used was derived from a fas-mutant B6-lpr/lpr mouse, since fasL expression can kill normal cells expressing the fas receptor (Muruve et al., Hum. Gene Ther. 8:955-963, 1997; Kang et al., Nature. Med. 3:738-743, 1997 and Larregina et al., Gene Ther. 5:563-568, 1998). Therefore, the expression of fasL would be extremely difficult to accomplish in cells derived from normal individuals.

There is a need for further adenoviral vector constructs for use in transgene delivery and expression to target cells in an individual, which reduce immune responses to the transgene as well as any other non-host related genes that might be expressed from the vector including viral genes. There is also a need for additional adenoviral vectors which enhance the persistence of a desired transgene expression in a host by decreasing or evading the host immune response to adenovirus.

### SUMMARY OF THE INVENTION

The invention is directed to a recombinant adenoviral vector comprising an adenovirus genome from which at least the adenovirus E1 region has been deleted, wherein at least one nucleic acid encoding an immunomodulatory molecule is inserted in said deletion in said vector, wherein said vector reduces or evades the host immune response from the cells of said individual. The recombinant adenoviral vector may also optionally contain a biologically active transgene, e.g., cystic fibrosis transmembrane protein, a biologically active human lysosomal enzyme or a tumor antigen. In contrast to prior art methods and vectors, the vectors of the present invention can deliver the nucleic acid encoding the immunomodulatory molecule to and express the nucleic acid encoding the immunomodulatory molecule in the cells of an individual to whom the vector has been delivered. No other component or factor is required to express or deliver the nucleic acid encoding the immunomodulatory molecule. This vector may be used for increasing the half-life of antigen-presenting cells for cancer immunotherapy applications by introducing into said cell an amount of said vector effective to increase the half life of said cells.

In a specific embodiment, the invention is directed to a recombinant adenoviral vector comprising an adenovirus genome from which at least the adenovirus E1 region and a second region of the adenovirus genome have been deleted, wherein the fasL gene is inserted into one deleted region of said vector and a transgene that codes for a molecule that inhibits apoptosis or CTL lysis is inserted into the second deleted region of said vector and wherein the vector is taken up by the cells and the fasL gene and transgene inhibiting apoptosis or CTL lysis are expressed. The vector may be used for inducing tolerance to adenovirus antigens or transgene products expressed by the vector in antigen-presenting cells of an individual to whom the vector has been administered. In a most specific embodiment, the vector comprising the fasL gene is inserted into the deleted E1 region and the baculovirus p35 gene or P-I-9 gene is inserted into the deleted E3 region of the adenovirus genome.

The invention further relates to a recombinant adenoviral vector, comprising an adenovirus genome from which the adenovirus E1 region and at least one other region of the adenovirus genome have been deleted, wherein a transgene of interest is inserted in one deletion and at least one nucleic acid encoding an immunomodulatory molecule is inserted in the other deletion, wherein said vector can deliver and express the transgene in cells of an individual to whom the vector is administered, and wherein said vector reduces or evades the host immune response from the cells of said individual. As a result, the vectors of the invention, which carry both a transgene of interest and a nucleic acid encoding an immunomodulatory molecule, facilitate persistence of the adenoviral vector and transgene expression in cells and tissues to which the vector has been administered. The invention is also directed to methods of using the vectors to provide a transgene to target cells of an individual to obtain transgene expression therein and an alteration in the phenotype of the target cells. The present invention provides a solution to the problem of adverse host immune response to adenoviral vectors without the removal of viral genes from the vectors that are necessary for successful administration and expression of transgenes. A further advantage of the invention is reduce significantly or to obviate any need for immunosuppression of the host in order to reduce the immune response to adenoviral vectors.

### BRIEF DESCRIPTION OF THE FIGURES

FIG. 1 shows an overview of the pAd_{vantage} system.

FIG. 1A shows the adenoviral cloning vector pAd_{vantage} and its construction.

FIG. 1B shows the shuttle vector pSV2-ICEU I.

FIG. 2 shows a map of the plasmid, pCMV.

FIG. 3 shows the expression cassette of the adenoviral genome containing the CFTR and p35 gene.

FIG. 4 shows a map of the plasmid, pAd/E4+/E3Δ2.9.

FIG. 5 shows the results of the ALT assay.

FIG. 6 shows the results of the AST assay.

FIG. 7 shows the expression cassette of the adenoviral genome containing the alpha-galactosidase and P-I-9 gene.

FIG. 8 shows the expression cassette of the adenoviral genome containing the gp100 and P-I-9 gene.

FIG. 9 shows the expression cassette of the adenoviral genome containing the fasL and p35 gene.

FIG. 10 shows the plasmid pAd2/E1-R.

FIG. 11 shows the plasmid pAd2/E1-R/FasL.

FIG. 12 shows the results of the LDH assay.

### DETAILED DESCRIPTION OF THE INVENTION

The adenoviral vectors in one embodiment of the present invention contain one or more nucleic acids encoding immunomodulatory molecule(s) which interact with the antiviral host immune apparatus to decrease or evade the immune response which destroys virions and infected cells. The immunomodulatory molecules can be inter alia, proteins, ribozymes, or antisense RNA. As a result, these vectors facilitate persistence of an adenoviral vector and desired transgene expression in cells and tissues of an individual to whom the vector has been administered. Circumvention of the host immune response is advantageous to the ability of the adenoviral vectors of the invention to effectively deliver transgenes repetitively into a host cell for expression of the transgene therein to obtain a phenotypic alteration in the cell correlated to the transgene product, as well as to induce tolerance to adenovirus antigens in antigen-presenting cells to whom the vector is administered. The present invention provides a solution to the current problem of adverse host immune response to adenoviral vectors, which has impeded the ability to repetitively administer the vectors to an individual. The invention may have the further advantage that removal of all viral genes from the vectors is not required and the need for host immunosuppression is greatly reduced or even eliminated. The present invention also includes methods for reducing or evading the host immune response using the vectors of the invention and methods for using such vectors to deliver a transgene to target cells and obtain expression therein.

In one embodiment, the vectors of the present invention comprise one or more nucleic acids encoding immunomodulatory molecules. In another embodiment, the vectors of the present invention also comprise a transgene.

Nucleic acids encoding immunomodulatory molecules which can be incorporated into the vectors of the present invention include, but are not limited to, baculovirus p35, fasL/CD95 ligand, CL P-I-9 protein.

The vectors of the present invention are based on replication-defective adenoviral vectors which can deliver a particular transgene to a cell, but which, for safety concerns, cannot replicate in the host. The adenoviral vectors of the invention are derived from the genome of various adenovirus serotypes, including but not limited to, adenovirus types 2, 4, 5, 6, 7 and 17, and in general, non-oncogenic serotypes. Preferably, the adenovirus is chosen from group C of the human adenoviruses, and is preferably adenovirus type 2, 5, or 6. The vectors of the invention may include genomic regions from different adenovirus serotypes.

Regions of the adenovirus genome which can be deleted in the vectors of the present invention to render them replication-incompetent include the E1 genomic region; other genomic regions from which deletions can be made include E2, E3 and E4 (Berkner, K., Curr. Top. Micro. Immunol. 158:39-66, 1992).

The nucleic acids encoding immunomodulatory molecules may be cloned into any recombinant adenoviral vector suitable for the delivery of a desired transgene to a recipient cell. One such vector comprises an adenovirus genome in which the E1 region and a 1.6 kb portion in the E3 region of the adenoviral genome from nucleotides 29292-30840 are deleted as disclosed in U.S. application Ser. No. 08/839,553. In such a vector, the adenovirus E4 region is preferably retained in the vector. In a preferred embodiment, this vector contains a deletion in the E1 genomic region of the virus which removes the coding sequences for the E1A protein required for autonomous replication and which may remove all or part of the E1B region of the virus genome. The protein IX coding sequences in the adenovirus genome may be retained in these vectors in order to optimize packaging capacity. In a particular embodiment, the protein IX coding sequences may be relocated from their position at the position of the E1 sequences to another location in the adenovirus in order to decrease the ability of these sequences to mediate recombination with homologous adenovirus sequences in packaging cell lines which can result in the generation of replication-competent adenovirus during vector production (U.S. Pat. Nos. 5,824,544 and 5,707,618; Hehir et al., J. Virol. 70:8459-8467, 1996). These vectors retain all or part of the adenovirus E2 genomic region. Modifications to the E3 region, such as truncations of the E3 coding sequence or deletions that remove particular open reading frames are permissible, providing that these alterations do not interfere with persistent expression of the nucleic acid encoding the immunomodulatory molecule or transgene. Preferably, the modifications to the E3 region retain the gene for the gp19K protein, due to its particular immunomodulatory role in preventing viral antigen presentation, thereby limiting the CTL response to adenovirus-infected cells (Wold et al., Trends Microbiol. 437-443, 1994). The retention of adenovirus genes for other immunomodulatory proteins such as 10.4K, 14.5K and 14.7 K is also within the scope of the modifications contemplated for the E3 region. Alternatively, all E3 genes can be deleted (nucleotides 27,971-30,937).

In a specific embodiment, the adenoviral vector is Ad2/CMV/E3Δ1.6 which contains the CMV promoter to which a nucleic acid encoding an immunomodulatory molecule may be operably linked and further contains an E1 deletion and a partial deletion of 1.6 kb from the E3 region (as disclosed in U.S. patent application Ser. No. 08/839,553). Specifically it contains a deletion in the E3 region which encompasses 1549 nucleotides from adenovirus nucleotides 29292 to 30840 (Roberts, R. J. et al., Adenovirus DNA, in Developments in Molecular Virology, W. Doerfler, ed., 8:1-15, 1986). These modifications to the E3 region in vector Ad2/CMV/E3Δ1.6 result in the following: (a) all the downstream splice acceptor sites in the E3 region are deleted and only mRNA (a) would be synthesized from the E3 promoter (Tollefson et al., J. Virol. 70:2296-2306, 1996; Tollefson et al., Virology 220:152-162, 1996); (b) the E3A poly A site has been deleted, but the E3B poly A site has been retained; ( c) the E3 gp19K (MHCI binding protein) gene has been retained; and (d) the E3 11.6K (Ad death protein) gene has been deleted. Alternatively, all E3 genes can be deleted (nucleotides 27,971-30,937).

In another embodiment, the vector is a partially-deleted adenoviral (termed “DeAd”) vector in which the majority of adenoviral early genes required for virus replication are deleted from the vector and placed within a producer cell chromosome under the control of a conditional promoter (see U.S. application Ser. Nos. 60/083,841 and 60/118,118). The deletable adenoviral genes that are placed in the producer cell include E1A/E1B, E2, E4 (only ORF6 and ORF6/7 need be placed into the cell), pIX and pIVa2. E3 may also be deleted from the vector, but since it is not required for vector production, it can be omitted from the producer cell. The adenoviral late genes, normally under the control of the major late promoter (MLP), are present in the vector, but the MLP has been replaced by a conditional promoter. Conditional promoters suitable for use in DeAd vectors and producer cell lines include the dimerizer gene control system, based on the immunosuppressive agents FK506 and rapamycin, the ecdysone gene control system and the tetracycline gene control system (tet on/tet off).

In another embodiment, the adenovirus E4 genomic region may be provided as a full length sequence or portions of the E4 region or individual open reading frames may be used which function analogously to the full-length sequence to promote persistent expression of the transgene contained in the transcription unit. Where individual open reading frames of the E4 region are used to prevent transcriptional down-regulation of the transgene, such genes may be placed under the control of the native EH promoters, or, alternatively, may be placed under the control of heterologous promoters. In a specific embodiment, the vector is Ad2E4ORF6 (U.S. Pat. No. 5,670,488; Armentano et al., Human Gene Therapy 6:1343-1353, 1995). The vector may also be chosen from vectors disclosed in PCT publication no. WO98/46781.

The nucleic acid encoding an immunomodulatory molecule or the transgene can be operably linked to expression control sequences, including but not limited to, a viral or non-viral promoter, in order to insure high level expression. As used herein, the phrase “operatively linked” refers to the functional relationship of a polynucleotide/transgene with regulatory and effector sequences of nucleotides, such as promoters, enhancers, transcriptional and translational stop sites, and other signal sequences. For example, operative linkage of a nucleic acid to a promoter refers to the physical and functional relationship between the polynucleotide and the promoter such that transcription of DNA is initiated from the promoter by an RNA polymerase that specifically recognizes and binds to the promoter and wherein the promoter directs the transcription of RNA from the polynucleotide. Such promoters include, but are not limited to, the cytomegalovirus (CMV) or Rous sarcoma virus long terminal repeat (RSV-LTR) viral promoters, as well as non-viral promoters, such as the PGK promoter. Tissue-specific promoters can also be used so that the nucleic acid encoding the immunomodulatory molecule or transgene is expressed only in desired therapeutically relevant cells. The nucleic acid encoding the immunomodulatory molecule or transgene can also be operably linked to its native promoter. A suitable viral or nonviral polyadenylation element, e.g., the SV40 or BGH polyA signal, can be engineered at the terminus of the gene. Alternatively, an adenoviral or native polyA signal can be used.

If the vector contains two nucleic acid encoding immunomodulatory molecules or a transgene and a nucleic acid encoding an immunomodulatory molecule, the nucleic acid (s) encoding the immunomodulatory molecule(s) and/or the desired transgene(s) may be expressed from different promoters in order to optimize specific expression of each type of gene. For example, the transgene may be placed under the control of a constitutive promoter, and the nucleic acid encoding the immunomodulatory molecule is placed under the control of an inducible promoter. In this manner, separate regulation of expression allows the functions of the vector to be specifically controlled. Alternatively, where the transgene and the nucleic acid encoding the immunomodulatory molecule are adjacent, they may be placed under the control of the same expression control sequences and simultaneous expression of both genes will be achieved.

In a preferred embodiment of the present invention (schematically illustrated in Example 4), a nucleic acid encoding an immunomodulatory molecule is inserted into the E1-deleted region of the adenoviral vector and a nucleic acid encoding a second immunomodulatory molecule is inserted into the E3-deleted region of the vector. Alternatively, the nucleic acid encoding the immunomodulatory molecule can be cloned into any site in the adenoviral vector, so long as there is no interference with the transcription of neighboring genes. A vector genome size that does not exceed 105-108% of the wild-type adenovirus genome length can be packaged, so that the desired transgene and nucleic acid encoding an immunomodulatory molecule which are inserted into the adenoviral vectors of the invention can be chosen with this guideline for optimal packaging. One or more nucleic acids coding for immunomodulatory molecules can be inserted into recombinant adenoviral vectors of the invention in any site of the genome.

To create the recombinant adenoviral vectors of the invention which contain a transgene of interest and a nucleic acid encoding an immunomodulatory molecule, a plasmid containing the desired nucleic acid encoding the immunomodulatory molecule inserted into an adenovirus genomic fragment is co-transfected with a linearized viral genome derived from an adenoviral vector of interest and containing the desired transgene into a recipient cell under conditions whereby homologous recombination occurs between the vector genomic fragment containing the nucleic acid encoding the immunomodulatory molecule and the vector containing the transgene. As a result, the gene encoding the immunomodulatory molecule is inserted into the adenoviral vector genome at the site in which it was cloned into the plasmid. This creates the recombinant adenoviral vector carrying the gene encoding the immunomodulatory molecule and the desired transgene. After homologous recombination has occurred, viral plaques are identified and isolated. The plaques are expanded and recombinant adenoviral vectors, now containing the nucleic acid encoding the immunomodulatory molecule and the transgene of interest, are identified by, for example, restriction enzyme analysis or PCR amplification of viral genomic DNA. Recombinant virus is then subjected to plaque purification in an appropriate cell line. Purified virus can be used to generate a vector stock.

To create the recombinant adenoviral vectors of the invention which contain a transgene of interest and a nucleic acid encoding an immunomodulatory molecule(s), a plasmid containing the desired nucleic acid encoding the immunomodulatory molecule inserted into an adenovirus genomic fragment is co-transfected with a linearized viral genome derived from an adenoviral vector of interest and containing the desired transgene into a recipient cell under conditions whereby homologous recombination occurs between the genomic fragment containing the nucleic acid encoding the immunomodulatory molecule and the vector containing the transgene. As a result, the nucleic acid encoding the immunomodulatory molecule is inserted into the adenoviral vector genome at the site in which it was cloned into the plasmid. This creates the recombinant adenoviral vector carrying the nucleic acid encoding the immunomodulatory molecule and the desired transgene. After homologous recombination has occurred, viral plaques are identified and isolated. The plaques are expanded and recombinant adenoviral vectors, now containing the nucleic acid encoding the immunomodulatory molecule and the transgene of interest, are identified by, for example, restriction enzyme analysis or PCR amplification of viral genomic DNA. Recombinant virus is then subjected to plaque purification in an appropriate cell line. Purified virus can be used to generate a vector stock.

Where the adenoviral vector of the invention is, preferably, a replication-defective vector, vector stocks may be produced using cell lines which contain the complementing regions of the adenovirus genome to allow replication and packaging of the recombinant vector genome so as to create a vector stock. Cell lines which can be used to generate the vectors of the invention include the 293 cell line, for example, where an E1-deleted vector is used, and can be any suitable complementing cell line when a replication-defective adenovirus is used to create the vector. Alternatively, vector stocks of replication defective vectors may be produced using helper viruses to supply the complementing adenovirus genes.

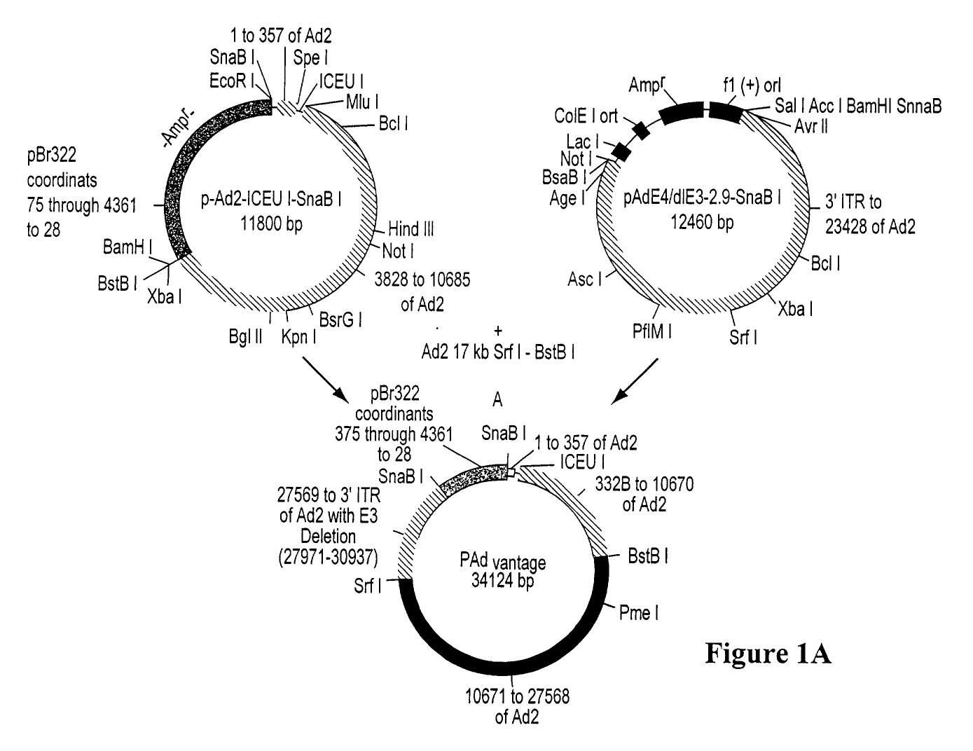

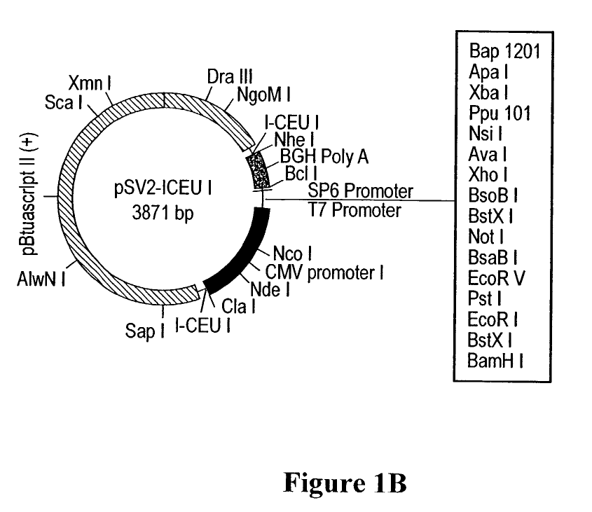

The recombinant adenoviral vectors may also obtained using the pAd_{vantage} rapid cloning system (Souza and Armentano, 1999, BioTechniques 26:502-508). The system is based on manipulating the full-length adenovirus genome as a stable plasmid in E. coli using intron-encoded endonucleases. These intron-encoded endonucleases cut their recognition sequences, which range from 15-39 bp, with high specificity. Three steps are involved. The transgene and nucleic acid encoding the immunomodulatory molecule is cloned into the shuttle vector pSV2-ICEU I (see FIG. 1B). It is then subcloned from pSV2-ICEU I by I-Ceu I digestion into the I site of the viral vector pAd_{vantage} (see FIG. 1A). The pAd_{vantage}-based recombinant adenoviral vector is cleaved with SnaBI to expose the inverted terminal repeats and is then transfected into 293 cells for generation of virus. Viral plaques are visible within seven to ten days. The total time to generate a recombinant virus is about three weeks.

The vectors of the present invention can be tested in vitro for expression of the nucleic acid encoding an immunomodulatory molecule prior to in vivo administration by infecting appropriate cell lines with the vector. Such cell lines include, but are not limited to, 293 cells (Graham et al., J. Gen. Virol. 36:59-71, 1977), A549 cells (human lung carcinoma, -ATCC accession number CCL-185) or normal human bronchial epithelial cells (NHBE). The expression of the nucleic acid encoding the immunomodulatory molecule in the vector can be monitored by any known technique for protein detection, e.g., immunoprecipitation, Western blots, ELISA assays or other methods known to those skilled in the art.

The determination of the host immune response includes, inter alia, identifying specific cellular responses, e.g., the generation of CTLs to adenovirus infected cells. However, the immune response is also characterized by tissue-damaging phenomena such as the infiltration of CTLs into an infected site. When, for example, the lung is a site of adenoviral vector administration, e.g., for administering a nucleic acid encoding CFTR to cells of an individual with cystic fibrosis in order to obtain a functional chloride ion channel in such cells, an inflammatory immune response to vector administration can be detected by examination of the lungs for epithelial cell damage as well as peribronchial, perivascular and alveolar infiltration by CTLs (Ginsberg et al., Proc. Natl.Acad. Sci. 88:1651-1655, 1991; Yang et al., Nature Genetics 7:362369, 1994).

Other assays to determine the magnitude of the host immune response include T cell proliferation assays and CTL assays (Kaplan et al., Gene Therapy 3:117-127, 1996) from cells isolated from the peripheral blood or spleens of treated individuals (obtained, e.g., by spleen biopsy). The level of anti-adenovirus antibodies in serum and bronchial alveolar lavage (BAL) fluid can be measured to assess the host humoral response to the administered vector. Where the immunomodulation occurs by decreasing the level of a cellular cytokine, e.g., by decreasing synthesis using an immunomodulatory protein, ribozyme or antisense RNA, measurement of the cytokine level in the host before and after administration of the vector can provide an indication of effective immunomodulation.

The present invention includes a method of using the vectors of the present invention for inducing tolerance to adenovirus antigens in antigen-presenting cells of an individual to whom the vectors are administered comprising introducing into said antigen-presenting cells an amount of a vector comprising an adenovirus genome comprising nucleic acids encoding said adenoviral antigens, said adenovirus genome also comprising a deletion of the E1 region and a deletion in a second region, wherein the fasL gene is inserted into one deleted region and a nucleic acid encoding an apoptosis inhibitor is inserted into the other deleted region and wherein the vector is taken up by the cells and the fasL gene and the nucleic acid encoding the apoptosis inhibitor are expressed. Antigen-presenting cells are cells that stimulate the activation of cytotoxic T lymphocytes and CD4+ helper T cells. Suitable sources of antigen-presenting cells include but are not limited to whole cells, such as dendritic cells or macrophages and foster antigen-presenting cells. In one embodiment, the individual has an autoimmune disease. In another embodiment, the individual has a decreased level of cytotoxic T cells and decreased CD4+ helper cells. In yet another embodiment, the individual has had an organ transplant.

The present invention is also directed to a method of using the vector of the present invention for increasing the half-life of cells in which a recombinant adenoviral vector has been introduced, e.g., antigen-presenting cells comprising introducing into said cells an amount of a recombinant adenoviral vector comprising an adenovirus genome from which at least the adenovirus E1 region has been deleted, wherein at least one nucleic acid encoding an immunomodulatory molecule has been inserted in said deletion, wherein said vector is taken up by the cells and the immunomodulatory nucleic acid is expressed, wherein the expressed product is effective to increase the half-life of said antigen-presenting cells. This is particularly useful when the vector comprises a nucleic acid encoding a tumor associated antigen for immunotherapy, such as gp100, TRP-2 or MART-1 and p35 and/or P-I-9, wherein the benefit is to increase the half-life of antigen presenting cells in the patient and thereby increase the immune response to the tumor antigen in the patient.

The present invention also includes a method for providing a transgene to the cells of an individual and having the transgene expressed in said cells to produce a phenotypic alteration correlated with the transgene product comprising introducing into the cells a recombinant adenoviral vector, comprising an adenoviral genome from which the E1 region and at least one other region of the adenovirus genome has been deleted, wherein a transgene of interest has been inserted in one deletion and at least one nucleic acid encoding at least one immunomodulatory molecule has been inserted in the other deletion, wherein the vector is taken up by the cells and the transgene and nucleic acid encoding the immunomodulatory molecules are delivered, the transgene is expressed therein to produce the phenotypic alteration and said vector reduces or evades the host immune response to the vector or cell. Furthermore, the present invention includes a method for providing persistent expression of a transgene to the cells of an individual comprising administering a recombinant adenoviral vector, comprising an adenovirus genome from which the E1 region and at least one other region of the adenovirus genome has been deleted, wherein a transgene of interest is inserted in one deletion and at least one nucleic acid encoding an immunomodulatory molecule is inserted in the other deletion, wherein said vector can deliver the transgene and immunomodulatory nucleic acid to cells of an individual, and wherein said vector reduces or evades the host immune response from the cells of said individual and wherein the vector provides persistent expression of the transgene in the target cells and a phenotypic alteration correlated with the transgene product.

Transgenes that can be incorporated into the adenoviral vectors of the invention include, but are not limited to, those encoding for enzymes, hormones, growth factors, cytokines, antigens, antibodies, and such specific transgenes as CFTR, alpha-antitrypsin, soluble CD4, ADA, Herpes Simplex Virus thymidine kinase, the tumor antigens gp100, MART-1 and TRP-2 and any other genes that are recognized in the art. Transgenic nucleic acids encoding molecules such as ribozymes or antisense RNA can also be inserted into the vectors of the invention for delivery to and expression in the target cells of an individual.

In a specific embodiment, the vectors of the present invention comprise a transgene that encodes a human lysosomal enzyme, such as human a-galactosidase. These enzymes may be delivered to cells deficient therein. Such vectors may be used to provide nucleic acids encoding specific lysosomal hydrolases, the deficiencies of which have been associated with various storage disorders. Table I lists the lysosomal storage diseases and associated enzymatic defects.

TABLE I
Lysosomal storage diseases and associated enzymatic defects

| Disease | Enzymatic Defect |
|---|---|
| Pompe disease | acid a-glucosidase (acid maltase) |
| MPSI* (Hurler disease) | a-L-iduronidase |
| MPSII (Hunter disease) | iduronate sulfatase |
| MPSIII (Sanfilippo) | heparan N-sulfatase |
| MPS IV (Morquio A) | galactose-6-sulfatase |
| MPS IV (Morquio B) | acid β-galactosidase |
| MPS VII (Sly disease) | β-glucoronidase |
| I-cell disease | N-acetylglucosamine-1-phosphotransferase |
| Schindler disease | a-N-acetylgalactosaminidase |
| | (a-galactosidase B) |
| Wolman disease | acid lipase |
| Cholestrol ester storage | acid lipase |
| disease | |
| Farber disease | lysosomal acid ceramidase |
| Niemann-Pick disease | acid sphingomyelinase |
| Gaucher disease | β-glucosidase (glucocerebrosidase) |
| Krabbe disease | galactosylceramidase |
| Fabry disease | a-galactosidase A |
| GM1 gangliosidosis | acid β-galactosidase |
| Galactosialidosis | β-galactosidase and neuraminidase |
| Tay-Sach's disease | hexosaminidase A |
| Sandhoff disease | hexosaminidase A and B |

*MPS = mucopolysaccaridosis

The vectors of the invention may be targeted to specific cells by linking a targeting molecule to the vector (as disclosed in PCT/US99/02680 filed Feb. 8, 1999). A targeting molecule is any agent that is specific for a cell or tissue type of interest, including for example, a ligand, antibody, sugar, receptor, or other binding molecule. The ability of targeted vectors renders invention vectors particularly useful in the treatment of lysosomal storage disorders. For example, including a targeting molecule, such as VEGF or an antibody to a VEGF receptor can provide targeting to vascular endothelial cells in individuals with Fabry's disease.

In addition, viral vectors, especially adenoviral vectors that have been complexed with a cationic amphiphile, such as a cationic lipid as described above, polyL-lysine (PLL), and diethylaminoethyldextran (DEAE-dextran) provide increased inefficiency of viral infection of target cells (See, e.g., WO98/22144). Other cationic amphiphiles useful for complexing with and facilitating the transfer of vectors of the invention are those lipids which are described in WO96/18372. In addition, since repeat administration of a viral vector can result in an immune response to the vector, thereby limiting its effectiveness in delivering the gene to affected cells, adenovirus and other viral vectors may be polymer-modified, e.g., complexed with polyethylene glycol (PEG), to reduce viral immunogenicity and allow for repeat administration of the vector (See, e.g., WO98/44143). Alternatively, the vector may be administered with an agent to reduce the immune response to repeated vector administration. In addition, combinations of the above approaches may be used.

Transfer of the transgene to the target cells by invention vectors can be evaluated by measuring the level of the transgene product in the target cell and correlating a phenotypic alteration associated with transgene expression. For example, expression of a CFTR transgene in target cells from an individual with cystic fibrosis is correlated with production of a functional chloride ion channel in such cells that may be measured by techniques known in the art. The level of transgene product in the target cell directly correlates with the efficiency of transfer of the transgene by invention vectors. Any method known in the art can be used to measure transgene product levels, such as ELISA, radioimmunoassay, assays using an fluorescent and chemiluminescent enzyme substrates.

Expression of the transgene can be monitored by a variety of methods known in the art including, inter alia, immunological, histochemical and activity assays. Immunological procedures useful for in vitro detection of the transgene product in a sample include immunoassays that employ a detectable antibody. Such immunoassays include, for example, ELISA, Pandex microfluorimetric assay, agglutination assays, flow cytometry, serum diagnostic assays and immunohistochemical staining procedures which are well known in the art. An antibody can be made detectable by various means well known in the art. For example, a detectable marker can be directly or indirectly attached to the antibody. Useful markers include, for example, radionuclides, enzymes, fluorogens, chromogens and chemiluminescent labels.

For in vivo imaging methods, a detectable antibody can be administered to a subject and the binding of the antibody to the transgene product can be detected by imaging techniques well known in the art. Suitable imaging agents are.known and include, for example, gamma-emitting radionuclides such as ¹¹¹In, ^{99m}Tc, ⁵¹Cr and the like, as well as paramagnetic metal ions, which are described in U.S. Pat. No. 4,647,447. The radionuclides permit the imaging of tissues by gamma scintillation photometry, positron emission tomography, single photon emission computed tomography and gamma camera whole body imaging, while paramagnetic metal ions permit visualization by magnetic resonance imaging.

The adenoviral vectors of the present invention can be assayed for the ability to decrease or evade the host immune response upon administration of the vector to a suitable host and to determine the persistence of transgene expression in vivo using the vectors of the present invention using an animal model. Such a model may be chosen with reference to such parameters as ease of delivery, identity of transgene, relevant molecular assays and assessment of clinical status. Where the transgene encodes a protein whose lack is associated with a particular disease state, an animal model which is representative of the disease state may optimally be used in order to assess a specific phenotypic result and clinical improvement, e.g., Fabry Knockout mice (as disclosed in U.S. patent application Ser. No. 09/182,245, filed Oct. 29, 1998).

Relevant animals in which the transgene expression system may be assayed include, but are not limited to, mice, rats, monkeys and rabbits. Suitable mouse strains in which the transgene expression system may be tested include, but are not limited to, C3H, C57B1/6 (wild-type and nude) and Balb/c (available from Taconic Farms, Germantown, N.Y.).

Where it is desirable to assess the host immune response to vector administration, testing in immune-competent and immune-deficient animals may be compared in order to define specific adverse responses generated by the immune system. The use of immune-deficient animals, e.g., nude mice, may be used to characterize vector performance and persistence of transgene expression, independent of an acquired host response and to identify other determinants of transgene persistence.

In order to determine the persistence of these vectors in the host, one skilled in the art can assay for the presence of these vectors by any means which identifies the transgene or nucleic acid encoding the immunomodulatory molecule (and its expression), for example, by assaying for transgene or nucleic acid encoding the immunomodulatory molecule mRNA level by RT-PCR, Northern blot or SI analysis, or by assaying for transgene protein expression by Western blot, immunoprecipitation, or radioimmunoassay. Alternatively, the presence of vector or the desired transgene DNA sequences per se in the host can be determined by any technique that identifies DNA sequences including Southern blot or slot blot analysis, or other methods known to those skilled in the art. Where a vector contains a marker gene, e.g., lacZ coding for E. coli, β-galactosidase, the presence of the vector may be determined by these same assays or a specific functional assay that screens for the marker protein (e.g., X-gal). The persistence of a vector of the invention in the host can also be determined from the continued observation of the therapeutic benefit conferred by the administration of the vector containing the transgene, e.g., the improvement or stabilization of pulmonary function following administration of a vector containing the CFTR gene to an individual with cystic fibrosis. This can be accomplished using spirometry for pulmonary function tests (PFT). Demonstration of the restoration of chloride ion channel function in the adenoviral vector-treated cells of a CF patient can also be used to assess the persistence of the transgene CFTR (Zabner et al., J. Clin. Invest. 97:1504-1511, 1996).

The present invention also encompasses compositions containing the vectors of the invention which can be administered in an amount effective to deliver a desired transgene and/or nucleic acid encoding an immunomodulatory molecule and to reduce or evade the host immune response using the immunomodulatory molecule (protein, ribozyme, antisense RNA). The compositions can include physiologically acceptable carriers, including any relevant solvents. As used herein, “physiologically acceptable carrier” includes any and all solvents, dispersion media, coatings, antibacterial and antifungal agents, isotonic and absorption delaying agents, and the like. Except insofar as any conventional media or agent is incompatible with the active ingredient, i.e., the adenoviral vectors of the invention, its use in the compositions of the invention is contemplated.

Routes of administration for the compositions containing the adenoviral vectors include conventional and physiologically acceptable routes such as direct delivery to the target organ or tissue, intranasal, intravenous, intramuscular, subcutaneous, intradernal, oral and other parenteral routes of administration.

The invention is further directed to methods for using the compositions of the invention in vivo or ex vivo applications in which it is desirable to deliver one or more transgenes into cells using the adenoviral vectors of the invention so as to provide persistent expression of a transgene encoding a biologically active molecule therein. In vivo applications involve the direct administration of an adenoviral vector of the invention formulated into a composition to the cells of an individual. Ex vivo applications involve the transfer of the adenoviral vector directly to harvested autologous cells which are maintained in vitro, followed by readministration of the transduced cells to a recipient.

In a specific embodiment, the adenoviral vector is transfected into antigen-presenting cells. Suitable sources of antigen-presenting cells (APCs) include, but are not limited to, whole cells such as dendritic cells or macrophages and foster antigen-presenting cells.

Dendritic cells, which are the principal initiators of antigen-specific immune responses have several molecules on their surface that are critical for T-cell activation (reviewed in van Schooten et al., Mol. Medicine Today, pp. 254-260, June 1997). One approach for isolating dendritic cells involves isolating bone marrow precursor cells (CD34+) from blood and stimulating them to differentiate into dendritic cells. The patient must be treated with cytokines such as GM-CSF to boost the number of circulating CD34+ stem cells in the peripheral blood. The second approach for isolating dendritic cells is to collect the relatively large numbers of precommitted dendritic cells already circulating in the blood. Previous techniques for preparing mature dendritic cells from human peripheral blood have involved combinations of physical procedures such as metrizamide gradients and adherence/nonadherence steps (Freudenthal et al., Proc. Natl. Sci. U.S.A. 87:7698-7702), 1990; Percoll gradient separations (Mehta-Damani et al., J. Immunol. 153:996-1003 , 1994); and fluorescence activated cell sorting techniques (Thomas et al., J. Immunol. 151:6840-6852, 1993). The preferred methods for isolation and culturing of dendritic cells are described in Bender et al., J. Immun. Meth. 196:121-135, 1996 and Romani et al., J. Immun. Meth. 196:137-151 , 1996.

“Foster” antigen-presenting cells are cells from the human cell line 174xCEM.T2, referred to as T2 and contain a mutation in its antigen processing pathway that restricts the association of endogenous peptides with cell surface MHC class I molecules (Zweerink et al., J. Immuno. 150:1763-1771, 1993). This is due to a large homozygous deletion in the MHC class II region encompassing the genes TAP1, TAP2 LMP1 and LMP2 which are required for antigen presentation to MHC class I CD8+ CTLs. In effect, only “empty” MHC class I molecules are presented on the surface of these cells. Exogenous peptide added to the culture medium binds to these MHC molecules provided that the peptide contains the allele-specific binding motif.

Retroviral infection or transfection of T2 cells with specific recombinant MHC alleles allows for the redirection of the MHC restriction profile. Libraries tailored to the recombinant allele will be preferentially presented by them because the anchor residues will prevent efficient binding to the endogenous allele. In at least one case, the cell line 174x CEM.T2 was transfected with a mouse H-2Ld MHC allele which rendered the cells sensitive to an H-2Ld restricted CTL clone (Crumpacker et al., 1992, J. Immunol. 148:3004). This technique generates recombinant foster antigen-presenting cells (APCs) specific for any MHC restricted CTL for which the variable chain of the MHC allele is cloned.

Several cases have demonstrated that transfection of non-professional APCs with allogenic MHC alleles aids greatly in the immunogenicity of the recombinant cell line (Leong et al., 1994, Int. J. Cancer 59:212-216 and Ostrand-Rosenberg et al., 1991, Int. J. Cancer Suppl. 6:61-68). Specifically, immunosensitivity is proportional to the level of expression of the MHC proteins.

High level expression of MHC molecules makes the APC “more visible” to the CTLs. Expressing the MHC allele of interest in T2 cells using a powerful transcriptional promoter (e.g., the CMV promoter) results in a more reactive antigen-presenting cell (most likely due to a higher concentration of reactive MHC-peptide complexes on the cell surface). Since only one type of MHC allele will interact with a given library, the presence of or expression level of the endogenous allele will not compromise specificity if the library is designed to bind to the newly transfected allele.

Dosage of an adenoviral vector of the invention which is to be administered to an individual is determined with reference to various parameters, including the condition to be treated, the age, weight and clinical status of the individual and the particular molecular defect requiring the provision of a biologically active protein. The dosage is preferably chosen so that administration causes a specific phenotypic result, as measured by molecular assays or clinical markers described above. Dosages of an adenoviral vector of the invention which can be used for example in providing a transgene contained in a vector to an individual for persistent expression of a biologically active protein encoded by the transgene and to achieve a specific phenotypic result range from approximately 108 infectious units (I.U.) to 10¹¹ I.U. for humans.

It is especially advantageous to formulate parenteral compositions in dosage unit form for ease of administration and uniformity of dosage. “Dosage unit form” as used herein refers to physically discrete units suited as unitary dosages for the subjects to be treated, each unit containing a predetermined quantity of active ingredient calculated to produce the specific phenotypic result in association with the required physiological carrier. The specification for the novel dosage unit forms of the invention are dictated by and directly depend on the unique characteristics of the adenoviral vector used in the formulation and the limitations inherent in the art of compounding. The principal active ingredient (the adenoviral vector) is compounded for convenient and effective administration with the physiologically acceptable carrier in dosage unit form as discussed above.

Maximum benefit and achievement of a specific phenotypic result from the administration of an adenoviral vector of the invention to deliver one or more transgenes to an individual may require repeated administration. Such repeated administration may involve the use of the same adenoviral vector, or, alternatively, may involve the use of different vectors engineered to carry the same transgene but which are rotated in order to alter viral antigen presentation and decrease host immune response.

The practice of the invention employs, unless otherwise indicated, conventional techniques of recombinant DNA technology, protein chemistry, microbiology and virology which are within the skill of those in the art. Such techniques are explained fully in the literature. See, e.g., Current Protocols in Molecular Biology. Ausubel et al., eds., John Wiley & Sons, Inc., New York, 1995.

The invention is illustrated by reference to the following examples.

### EXAMPLES

### Example 1

Ad Vectors Expressing Baculovirus Protein p35

Construction of Ad/p35 Vectors

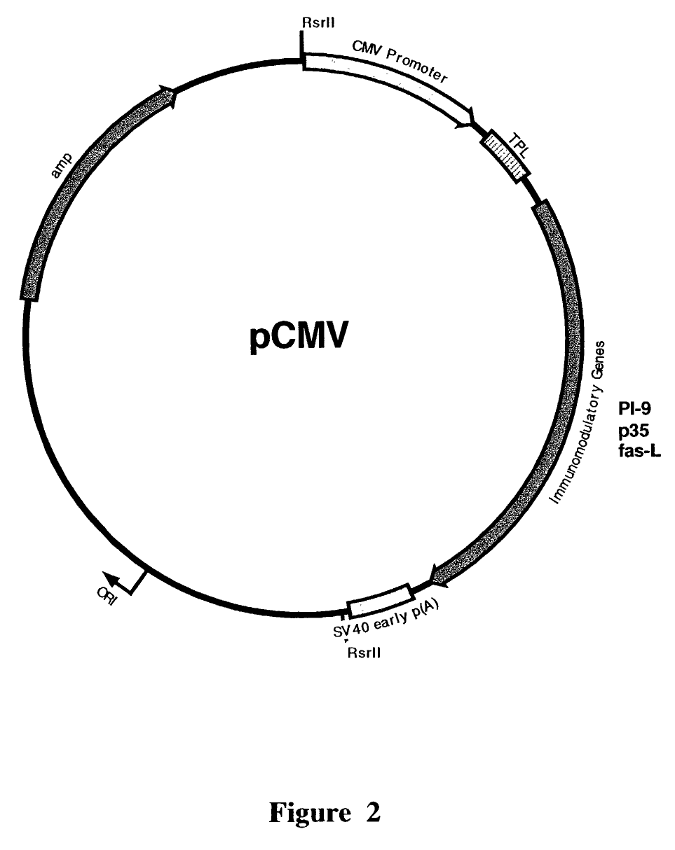

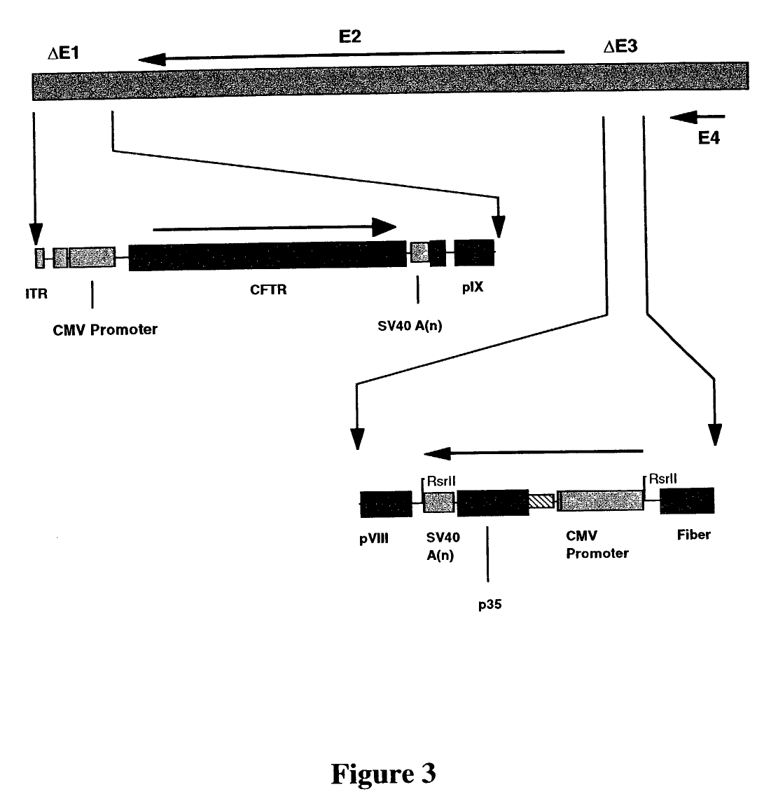

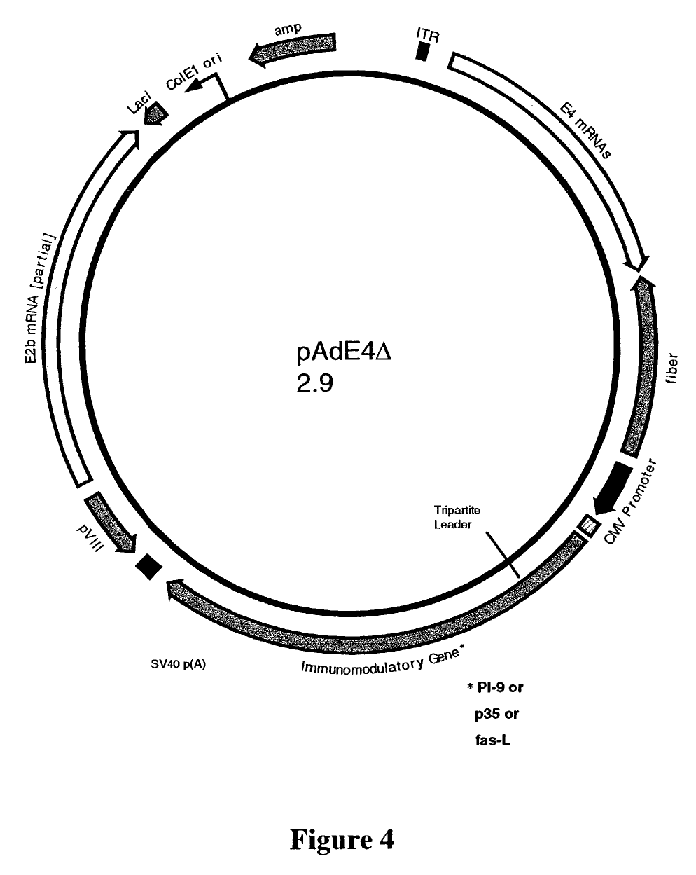

The baculovirus p35 gene is cloned by PCR from baculovirus genomic DNA and is inserted into an expression cassette plasmid pCMV (see FIG. 2) using the XhoI and NotI restriction sites (FIG. 3), so that the gene of interest is cloned behind the CMV promoter and uses the SV40 polyA sequence. The entire expression cassette is then cut out using the unique RsrII sites at either end of the cassette and cloned into the E3Δ2.9 deletion in an adenoviral plasmid, pAd/E4+/E3Δ2.9 (FIG. 4) which contains the right end of Ad2. This plasmid is then cotransfected with digested DNA from Ad2/CFTR5 so that homologous recombination occurs between the plasmid and the vector to yield vector Ad/CFTR/CMVp35 which can express CFTR and can also express baculovirus p35 from the CMV promoter.

In vitro TNF Cytotoxicity Assays

A549 and HeLa cells are tested in this assay. 2.5×10⁵ or 3×10⁵ cells per well are plated in 6-well dishes. Cells are infected with an Ad/CFYR/p35 vector or a control Ad2/CFTR vector at an multiplicity of infection (moi) of 100 to 150. At the same time, cells are co-infected with an Ad2/b-Gal2 reporter vector (used here merely as a reporter gene for cell death or survival) at an moi of 100 to 150. At 24 hours post infection, cells are treated with TNF@20 ng/ml and cycloheximide @15 to 20 mg/ml. Cells are fixed and stained for b-galactosidase expression 24 hours after addition of TNF. Fields of cells are counted and the presence of p35 prevented TNF mediated lysis is greater than 90% of cells. In control cells, >90% of the cells are lysed.

In vitro fas Ligand Killing Assays

Human (A549, HeLa), monkey (CV1) & mouse (SVBalb) cell lines were tested in this assay. 6-well dishes were plated with 0.9×10⁵ to 2×10⁵ cells/well. Cells were infected with an Ad/p35 vector or a control Ad vector at an moi of 100 to 150. 24 hours later, the same cells were infected with 50 moi of an Ad vector expressing both fas ligand and b-galactosidase reporter gene. Cells were fixed and stained for b-galactosidase expression at 24 hours after addition of the Ad/bgal/fasL vector. Fields of cells were counted and the presence of p35 prevented fas ligand-mediated killing in approximately 90% of cells.

In vivo Liver Toxicity Studies:

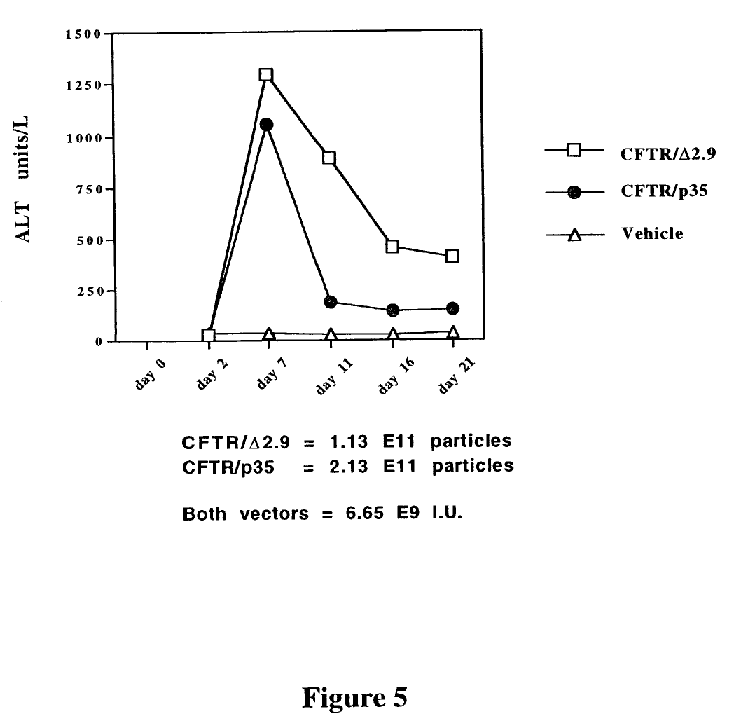

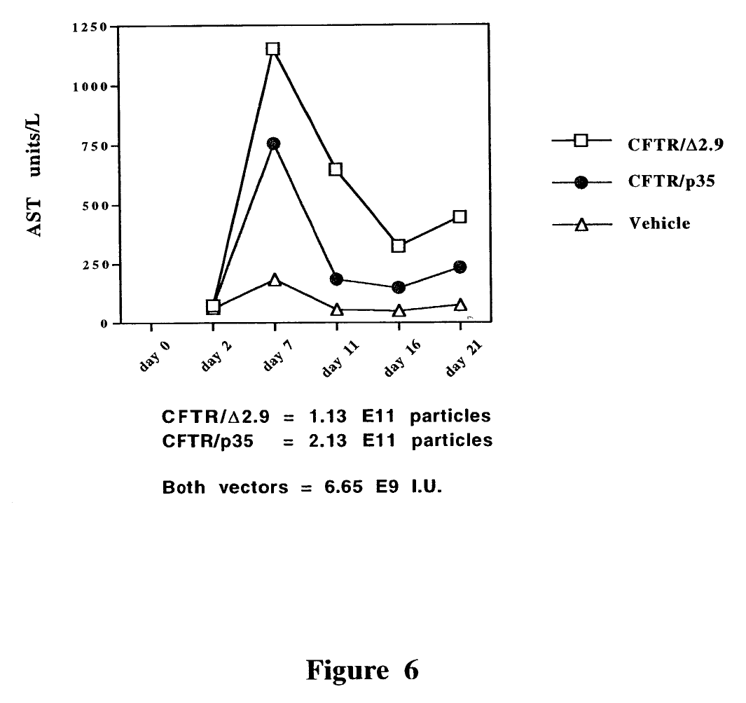

6.6×10⁹ I.U. of an Ad/CFTR/p35 vector or a control Ad/CFTR vector were injected intravenously into Balb/c mice to deliver the Ad vectors to the liver. To assess liver toxicity, the levels of serum transaminases ALT and AST were measured (Robins, Pathologic Basis of Disease, W. B. Saunders Company, 5th Edition) As shown in FIGS. 5 and 6, inclusion of p35 in the Ad vector reduced the toxicity caused by Ad vector delivered to the liver.

### Example 2

Ad Vectors Expressing Lymphocyte Proteinase Inhibitor P-I-9

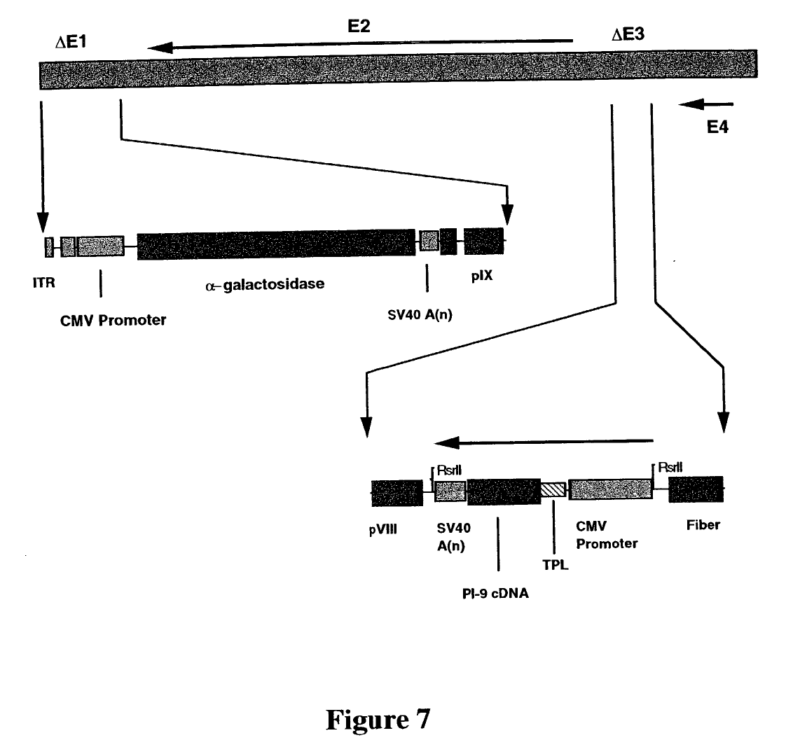

The P-I-9 cDNA is cloned by PCR from a human leukocyte quick-clone cDNA library (Clonetech Laboratories) and is inserted into the expression plasmid pCMV using the XhoI and NotI restriction sites (see FIG. 2), so that the cDNA is cloned behind the CMV promoter and used the SV40 polyA site. The entire expression cassette is then cut out using the unique RsrII sites at either end of the cassette and cloned into the E3Δ2.9 deletion in the pre-adenoviral plasmid, pAD/E4+/E3Δ2.9 (FIG. 4) which contains the right end of Ad2 including the fiber gene and the E4 region. This plasmid is then co-transfected into 293 cells with restricted DNA from an Ad2/CMV α-galactosidase from the E1 region and P-I-9 from the E3 region, both genes being driven by the CMV promoter (FIG. 7).

### Example 3

Prolonging the Half-life of Dendritic Cells for Cancer Vaccine Purpose

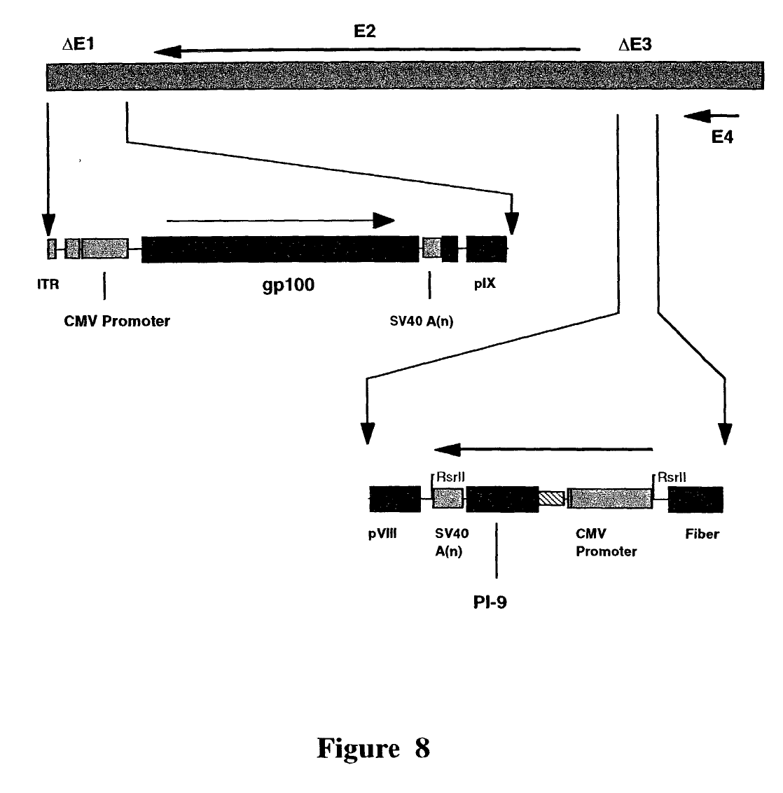

The baculovirus P-I-9 gene is cloned by PCR from baculovirus genomic DNA and is inserted into an expression cassette plasmid pCMV (see FIG. 2) using the XhoI and NotI restriction sites, so that the gp100 gene is cloned behind the CMV promoter and uses the SV40 polyA sequence. The entire expression cassette is then cut out using the unique RsrII sites at either end of the cassette and cloned into the E3Δ2.9 deletion in an adenoviral plasmid, pAd/E4+/E3Δ2.9 (FIG. 4) which contains the right end of Ad2. This plasmid is then cotransfected into 293 with digested DNA from an Ad2/CMV gp100 from the E1 region and P-I-9 from the E3 region, both genes being driven by the CMV promoter (FIG. 8). Dendritic cells are isolated from a patient and are exposed to the vector described in FIG. 8 to achieve expression of the tumor antigen gp100 and expression of P-I-9 within the dendritic cell. Such cells are subsequently reinfused into the patient from which they were isolated. Such cells provoke a cytotoxic T lymphocyte response to the tumor antigen which is beneficial to the patient. The presence of the P-I-9 gene and expression of the P-I-9 protein allows such cells to resist the attack of gp100-specific cytotoxic T lymphocytes thus allowing longer survivial of the cells in the patient and longer antigen presentiation in the patient as compared to similar cells modified to express only the tumor antigen but not the P-I-9 protein.

### Example 4

A FasL/p35 Vector

Construction of Ad/fasL/p35 Vector

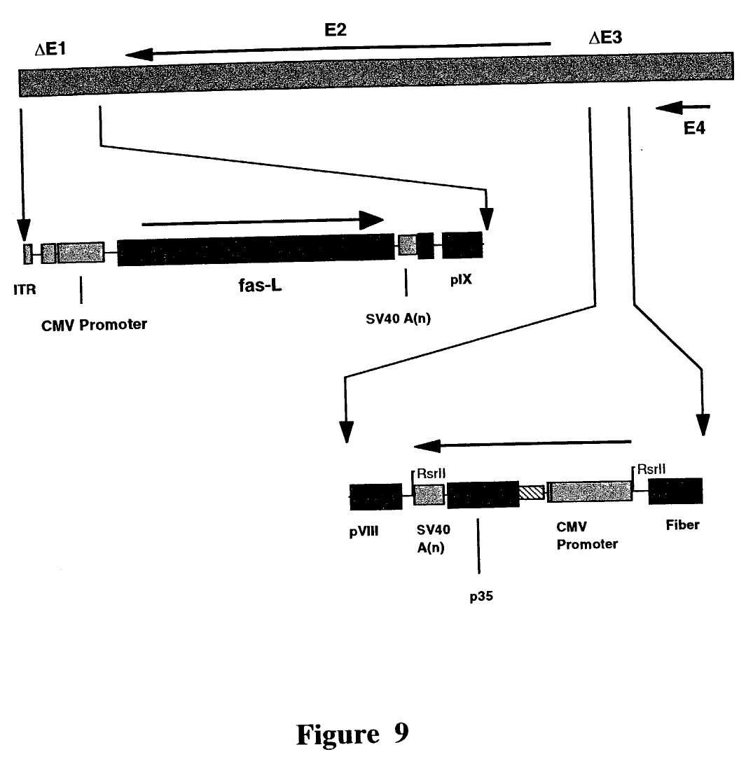

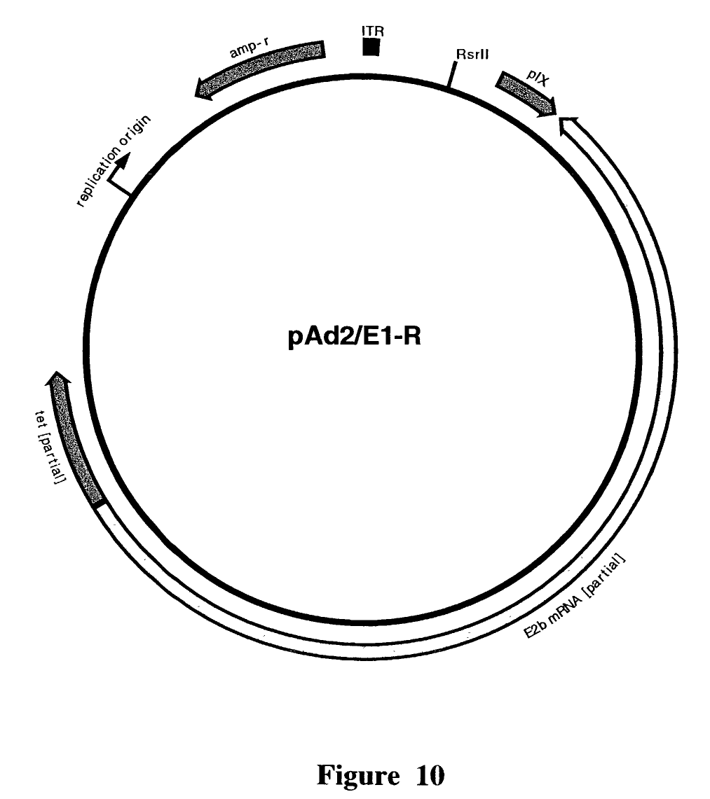

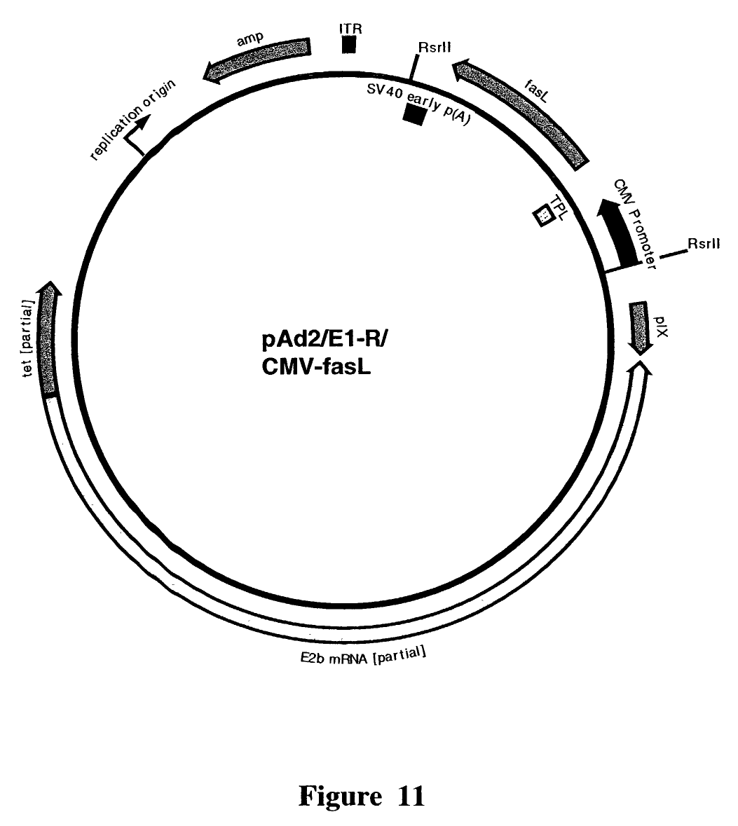

The mouse fas ligand cDNA is cloned by PCR from a mouse testis quick-clone cDNA library (Clonetech Laboratories) and is inserted into the expression plasmid, pCMV using the XhoI and NotI restriction sites (FIG. 2), so that the cDNA is cloned behind the CMV promoter and uses the SV40 polyA site. The entire expression cassette is then cut out using the unique RsrIl sites at either end of the cassette and cloned into the RsrII site in the E1 deleted (Ad2 nucleotides 358 to 3328 deleted) pre-adenoviral plasmid called pAdE1-R (see FIGS. 9 and 10) which contains the left end of Ad2 including the protein IX gene and E2B sequences. This plasmid is then cotransfected with restricted DNA from an Ad2/CFTR/p35 vector so that homologous recombination occurs between the plasmid and the Ad vector to yield Ad/fasL/p35 which expresses fasL from the E1 region and p35 from the E3 region, both genes being driven by the CMV promoter (FIGS. 9 and 11).

The Ad/fasL/p35 vector is produced in high titers of 10¹¹ I.U./ml in regular 293 cells used to produce E1 deleted Ad vectors. In comparison, titers of 10⁹ I.U./ml are obtained with other Ad vectors constitutively expressing fasL.

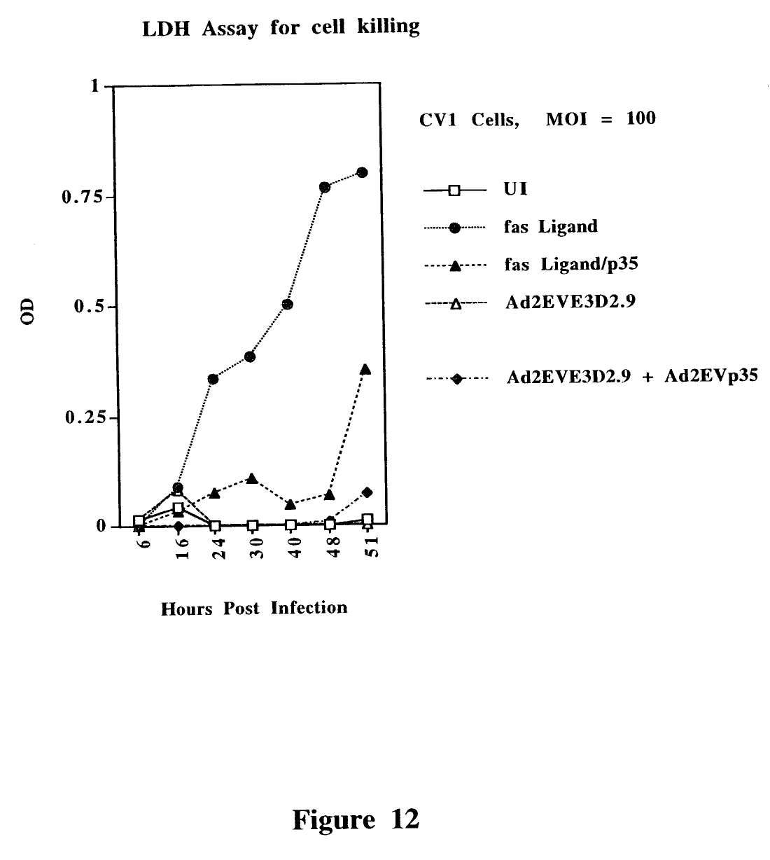

To determine if the Ad/fasL/p35 vector kills infected cells, a cell killing assay is performed on CV-1 cells using an LDH kit purchased from Promega. The results are shown in FIG. 12 and indicate that the Ad/fasL/p35 vector does not kill infected cells like the control Ad/fasL vector.

The invention described and claimed herein is not to be limited in scope by the specific embodiments herein disclosed, since these embodiments are intended as illustrations of several aspects of the invention. Any equivalent embodiments are intended to be within the scope of this invention. Indeed, various modifications of the invention in addition to those shown and described herein will become apparent to those skilled in the art from the foregoing description. Such modifications are also intended to fall within the scope of the appended claims.

Various references are cited herein, the disclosures of which are incorporated by reference in their entireties.

What is claimed is:

1. A recombinant adenoviral vector comprising an adenovirus genome from which at least the adenovirus E1 region has been deleted, wherein at least one nucleic acid encoding an immunomodulatory molecule is inserted in said deletion, wherein said vector delivers the nucleic acid encoding the immunomodulatory molecule to and expresses the nucleic acid encoding the immunomodulatory molecule in the cells of an individual to whom the vector is delivered and wherein said vector reduces or evades the host immune response from the cells of said individual.

2. The vector of claim 1, in which the nucleic acid encoding an immunomodulatory molecule is selected from the group consisting of the genes for baculovirus p35, fasL/CD95 ligand and cytotoxic lymphocyte serpin proteinase inhibitor 9.

3. The vector of claim 1, in which the nucleic acid encoding the immunomodulatory molecule is operably linked to expression control sequences.

4. The vector of claim 3, in which the expression control sequences include the cytomegalovirus immediate early promoter.

5. The vector of claim 1, from which a 1.6 kb portion encompassing nucleotides 29292-30840 in the E3 region of the adenovirus genome have been deleted.

6. The vector of claim 5, in which the vector comprises two nucleic acids encoding immunomodulatory molecules, one inserted into the deletion in the E1 region and the other inserted into the deletion in the E3 region.

7. The vector of claim 5, in which the vector comprises the fasL gene inserted into the deletion in the E1 region and the baculovirus p35 gene inserted into the deletion of the E3 region.

8. The vector of claim 1, from which about a 3.0 kb portion encompassing nucleotides 27971-30937 in the E3 region of the adenovirus genome have been deleted.

9. A method for inducing tolerance to at least one adenovirus antigen and/or transgene product in cells of an individual to whom a recombinant adenoviral vector comprising (a) said adenoviral antigen and (b) further comprising an adenovirus genome from which the adenovirus E1 region and a second region of the adenovirus genome has been deleted, wherein the fasL gene is inserted into one deleted region and a transgene that codes for a molecule that inhibits apoptosis is inserted into the second deleted region and wherein the vector is taken up by the cells and the fasL gene and transgene inhibiting apoptosis are expressed, has been admninistered, comprising administering said recombinant adenoviral vector to an individual in an amount effective to induce tolerance to said adenovirus antigen in an individual to whom the vector has been administered.

10. The method of claim 9, in which tolerance is induced in antigen-presenting cells.

11. The method of claim 9, wherein said vector is transferred to harvested autologous cells maintained in vitro and said cells are administered to said individual.

12. The method of claim 9, wherein said vector is directly administered to said individual.

13. The method of claim 8, in which the gene inhibiting apoptosis is the baculovirus p35 gene or the cytotoxic lymphocyte serpin proteinase inhibitor 9.

14. A method for increasing the half-life of cells that have taken up a recombinant adenoviral vector, comprising introducing into said cells a recombinant adenoviral vector comprising an adenovirus genome from which the adenovirus E1 region has been deleted, wherein at least one nucleic acid encoding an immunomodulatory molecule is inserted in said deletion, wherein said vector is taken up by the cells and the immunomodulatory nucleic acid is expressed in an amount effective to increase the half-life of said antigen-presenting cells.

15. The method according to claim 14, in which the cells are antigen-presenting cells.

16. The method according to claim 15, in which the antigen-presenting cells are dendritic cells.

17. A recombinant adenoviral vector comprising an adenovirus genome from which the adenovirus E1 region and at least one other region of the adenovirus genome has been deleted, wherein a transgene of interest has been inserted in one deletion and at least one at least one nucleic acid encoding an immunomodulatory molecule has been inserted in the other deletion, wherein said vector can deliver to and express the transgene in cells of an individual to whom the vector is administered, and wherein said vector reduces or evades the host immune response from the cells of said individual.

18. The vector of claim 17, wherein said transgene is a nucleotide sequence encoding a protein selected from the group consisting of a cystic fibrosis transmembrane protein, a biologically active human lysosomal enzyme, and a tumor antigen.

19. The vector of claim 17, wherein said transgene is a nucleotide sequence encoding a tumor antigen selected from the group consisting of gp100, MART-1 and TRP-2.

20. The vector of claim 17, in which the vector comprises an adenoviral genome from a 1.6 kb portion encompassing nucleotides 29292-30840 in the E3 region of the adenovirus genome have been deleted.

21. The vector of claim 20, in which the immunomodulatory molecule is selected from the group consisting of baculovirus p35, fasL/CD95 ligand and cytotoxic lymphocyte serpin proteinase inhibitor 9.

22. The vector of claim 20, in which the transgene is inserted into the deleted E1 region and the nucleic acid encoding the immunomodulatory molecule is inserted into the deleted E3 region.

23. The vector of claim 17, from which about a 3.0 kb portion encompassing nucleotides 27971-30937 in the E3 region of the adenovirus genome have been deleted.

24. The vector of claim 17, in which the transgene is operably linked to expression control sequences.

25. The vector of claim 17, in which the nucleic acid encoding the immunomodulatory molecule is operably linked to expression control sequences.

26. The vector of claim 17, in which the transgene and the nucleic acid encoding the immunomodulatory molecule are operably linked to the same expression control sequence.

27. The vector of claim 26, in which the expression control sequence is a cytomegalovirus promoter.

28. A method for providing a transgene to the cells of an individual and having the transgene expressed in said cells to produce a phenotypic alteration comprising, introducing into the cells a recombinant adenoviral vector, comprising an adenovirus genome from which the adenovirus E1 region and at least one other region of the adenovirus genome have been deleted, wherein a transgene of interest is inserted in one deletion and at least one at least one nucleic acid encoding an immunomodulatory molecule is inserted in the other deletion, wherein the vector is taken up by the cells and the transgene and nucleic acid encoding the immunomodulatory molecule are delivered, the transgene is expressed therein and said vector reduces or evades the host immune response to the vector or cell.

29. A method for providing persistent expression of a transgene to target cells of an individual, comprising administering a composition comprising a recombinant adenoviral vector, comprising an adenovirus genome from which the adenovirus E1 region and at least one other region of the adenovirus genome have been deleted, wherein a transgene of interest is inserted in one deletion and at least one nucleic acid encoding an immunomodulatory molecule is inserted in the other deletion, wherein said vector can deliver the transgene and nucleic acid encoding the immunomodulatory molecule to cells of an individual, and wherein said vector reduces or evades the host immune response from the cells of said individual and a physiologically effective carrier in an amount effective to generate such persistent expression of the transgene to target cells of said individual.

30. A composition comprising a recombinant adenoviral vector comprising an adenovirus genome from which at least the adenovirus E1 region have been deleted, wherein at least one nucleic acid encoding an immunomodulatory molecule is inserted at said deletion, wherein said vector delivers the nucleic acid encoding the immunomodulatory molecule to and expresses the nucleic acid encoding the immunomodulatory molecule in the cells of an individual to whom the vector is delivered and wherein said vector reduces or evades the host immune response from the cells of said individual and a physiologically effective carrier.

31. A composition comprising a recombinant adenoviral vector, comprising an adenovirus genome from which the adenovirus E1 region and at least one other region of the adenovirus genome have been deleted, wherein a transgene of interest is inserted in one deletion and at least one at least one nucleic acid encoding an immunomodulatory molecule is inserted in the other deletion, wherein said vector can deliver to and express the transgene in cells of an individual to whom the vector is administered, and wherein said vector reduces or evades the host immune response from the cells of said individual and a physiologically effective carrier.
